(19) United States
(12) Patent Application Publication
(10) Pub. No.: US 2023/0372643 A1
(43) Pub. Date: Nov. 23, 2023

(54) DEVICES, ASSEMBLIES, AND METHODS FOR DELIVERING AGENTS

(71) Applicant: Boston Scientific Scimed Inc., Maple Grove, MN (US)

(72) Inventors: Ra Nam, Lawrence, MA (US); Adam Gregory McDERMOTT, Lincoln, MA (US); Andrew PIC, Northboro, MA (US); John B. GOLDEN, Norton, MA (US)

(73) Assignee: Boston Scientific Scimed Inc., Maple Grove, MN (US)

(21) Appl. No.: 18/319,750

(22) Filed: May 18, 2023

Related U.S. Application Data
(60) Provisional application No. 63/343,833, filed on May 19, 2022.

(51) Int. Cl. A61M 11/06; A61M 39/22; A61M 11/00

(52) U.S. Cl. CPC A61M 11/06; A61M 39/22; A61M 11/001; A61M 2202/064

(57) ABSTRACT

A valve assembly that includes an inlet that is in fluid communication with an enclosure of a medical device, an outlet that is in fluid communication with a delivery conduit of the medical device, and a body having a channel that is in fluid communication with a source of fluid. The body is configured to move relative to the inlet and outlet to selectively fluidly couple the channel with the enclosure and delivery conduit. In a first position of the body, the channel is misaligned with at least one of the inlet or the outlet, such that the delivery conduit is not in fluid communication with at least one of the enclosure or the source of fluid. In a second position of the body, the channel is aligned with the inlet and outlet such that the delivery conduit is in fluid communication with the enclosure and the source of fluid.

### CROSS-REFERENCE TO RELATED APPLICATIONS

This application claims the benefit of priority of U.S. Provisional Patent Application No. 63/343,833, filed May 19, 2022, the entirety of which is incorporated herein by reference.

### TECHNICAL FIELD

Various aspects of this disclosure relate generally to devices and methods for delivering agents. More specifically, in embodiments, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines.

During an endoscopic procedure, a user inserts a sheath of an endoscope into a body lumen of a patient. The user utilizes a handle of the endoscope to control the endoscope during the procedure. Tools are passed through a working channel of the endoscope via, for example, a port in the handle, to deliver treatment at the procedure site near a distal end of the endoscope. The procedure site is remote from the operator.

To achieve hemostasis at the remote site, a hemostatic agent may be delivered by a device inserted into the working channel of the endoscope. Agent delivery may be achieved through systems that may be manually operated, for example. Such systems, however, may require numerous steps or actuations to achieve delivery, may not achieve a desired rate of agent delivery or a desired dosage of agent, may result in the agent clogging portions of the delivery device, may result in inconsistent dosing of agent, or may not result in the agent reaching the treatment site deep within the GI tract. The current disclosure may solve one or more of these issues or other issues in the art.

### SUMMARY

Each of the aspects disclosed herein may include one or more of the features described in connection with any of the other disclosed aspects.

According to an example, a valve assembly for a medical device includes: an inlet that is in fluid communication with an enclosure of the medical device, wherein the enclosure stores an agent; an outlet that is in fluid communication with a delivery conduit of the medical device; and a body having a channel that is in fluid communication with a source of fluid, wherein the body is configured to move relative to the inlet and the outlet to selectively fluidly couple the channel with the enclosure and the delivery conduit; wherein, in a first position of the body, the channel is misaligned with at least one of the inlet or the outlet, such that the delivery conduit is not in fluid communication with at least one of the enclosure or the source of fluid; and wherein, in a second position of the body, the channel is aligned with the inlet and the outlet such that the delivery conduit is in fluid communication with the enclosure and the source of fluid.

Any of the valve assemblies described herein may include any of the following features. In the first position, the channel is positioned in a transverse alignment relative to an axis extending between the inlet and the outlet. In the second position, the channel is positioned in a parallel alignment relative to an axis extending between the inlet and the outlet. The channel is in fluid communication with the source of fluid when the body is in the first position and the second position. The body is configured to guide the fluid through the channel and into the enclosure via the inlet to agitate the agent within the enclosure when in the first position. The body is configured to guide a mixture of the fluid and the agent from the enclosure into the channel via the inlet, and the channel is configured to guide the mixture into the delivery conduit via the outlet. The body includes an insert positioned within the channel, the insert including a porous mesh. The insert is configured to inhibit the agent from moving through the insert towards the source of fluid, and to permit the fluid to pass through the insert. The channel is a first channel, and wherein the body includes a second channel that is in fluid communication with the first channel, the second channel having a length that is less than a length of the first channel. In the first position and the second position of the body, the second channel is: misaligned with the outlet; in fluid communication with the source of fluid; and not in fluid communication with the inlet. In a third position of the body, the second channel is aligned with the outlet such that the delivery conduit is in fluid communication with the source of fluid via the second channel. In the third position of the body, the channel is misaligned with the inlet and the outlet, such that the delivery conduit is not in fluid communication with the enclosure via the first channel. The body includes an insert positioned within the second channel, and wherein the insert is configured to inhibit the agent from moving through the second channel and permit the fluid to pass through the second channel. Further including a housing that defines the inlet and the outlet, the housing is configured to receive the body, wherein a gap is formed between the housing and the body, the gap is positioned between the inlet and the outlet such that the delivery conduit is in fluid communication with the source of fluid via the gap when the body is in the first position. The gap is sized such that the agent cannot pass through the gap.

According to another example, a device for delivering an agent includes: an enclosure configured to store the agent, the enclosure having an inlet; a pressurized fluid source configured to store a pressurized fluid; a valve assembly, including a body having a channel, wherein the body is configured to move between a first position and a second position to selectively fluidly couple the pressurized fluid source to the enclosure via the channel; wherein, in the first position, the valve assembly is configured to misalign the channel from the inlet to inhibit the pressurized fluid from moving through the channel and delivering the agent out of the device; and wherein, in the second position, the valve assembly is configured to align the channel with the inlet to permit the pressurized fluid from moving through the channel and delivering the agent out of the device.

Any of the devices described herein may include any of the following features. The body includes an insert positioned within the channel that is configured to inhibit the agent from moving through the insert and permit the pressurized fluid to pass through the insert. The channel is a first channel, wherein the body includes a second channel that is in fluid communication with the first channel. In the first position and the second position of the body, the valve assembly is configured to inhibit the pressurized fluid from moving through the second channel and out of the device, and in a third position of the body, the valve assembly is configured to permit the pressurized fluid to move through the second channel and out of the device. The valve assembly includes a housing that is configured to receive the body, wherein a gap is formed between the housing and the body. When in the first position, the valve assembly is configured to permit the pressurized fluid to move out of the device via the gap while inhibiting delivery of the agent out of the device.

According to a further example, a method for delivering a fluid from a medical device, with the medical device including an enclosure for storing an agent, includes: moving a channel of a valve body to a first position that is aligned with an inlet and an outlet of the enclosure, thereby permitting delivery of a fluid through the channel and into the enclosure via the inlet to agitate the agent in the enclosure, and permitting delivery of the fluid and the agitated agent through the channel and toward a delivery conduit of the medical device via the outlet; and moving the channel to a second position that is misaligned with the inlet and the outlet, thereby inhibiting delivery of the fluid through the channel and into the enclosure via the inlet, and inhibiting delivery of the fluid toward the delivery conduit via the outlet.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device.

FIG. 2 shows a perspective view of an exemplary valve assembly of the delivery device of FIG. 1.

FIG. 3A shows a partial side view of the valve assembly of FIG. 2 in a first position.

FIG. 3B shows a partial side view of the valve assembly of FIG. 2 in a second position.

FIG. 4 shows a perspective view of another exemplary valve assembly of the delivery device of FIG. 1.

FIG. 5A shows a partial side view of the valve assembly of FIG. 4 in a first position.

FIG. 5B shows a partial side view of the valve assembly of FIG. 4 in a second position.

FIG. 5C shows a partial side view of the valve assembly of FIG. 4 in a third position.

FIG. 6 shows a perspective view of another exemplary valve assembly of the delivery device of FIG. 1.

FIG. 7 shows a perspective view of a movable body of the valve assembly of FIG. 6.

FIG. 8A shows a partial side view of the valve assembly of FIG. 6 in a first position.

FIG. 8B shows a partial side view of the valve assembly of FIG. 6 in a second position.

FIG. 9A shows a partial side view of the movable body of FIG. 7 in a first position.

FIG. 9B shows a partial side view of the movable body of FIG. 7 in a second position.

FIG. 10A shows a partial side view of another exemplary valve assembly of the delivery device of FIG. 1 in a first position.

FIG. 10B shows a partial side view of the valve assembly of FIG. 10A in a second position.

FIG. 10C shows a partial side view of the valve assembly of FIG. 10A in a third position.

FIG. 11 shows a partial side view of another exemplary valve assembly of the delivery device of FIG. 1.

### DETAILED DESCRIPTION

Embodiments of this disclosure relate to dispensing devices having valve assemblies for selectively releasing a pressurized fluid for delivering an agent (e.g., a powdered agent) to a site of a medical procedure. The valve assembly may include a movable body for fluidly coupling a pressurized medium source (e.g., a gas canister), from which the pressurized fluid (e.g., a gas) may be released, with an enclosure storing the agent. The agent may be received within the enclosure of the dispending device, and in selective fluid communication with the pressurized fluid through a channel of the movable body. Accordingly, when the channel is selectively moved into alignment with the enclosure by the valve assembly, the pressurized fluid received within the channel from the pressurized fluid source may travel toward and enter the enclosure to agitate the agent prior to delivery to a target site of the medical procedure. Aspects of the dispensing device and valve assembly, such as the movable body and channel, may facilitate a fluidization of the agent with the flow of pressurized fluid prior to the agent being delivered, which may assist in selectively controlling the flow of pressurized fluid to help to prevent or minimize clogging during delivery.

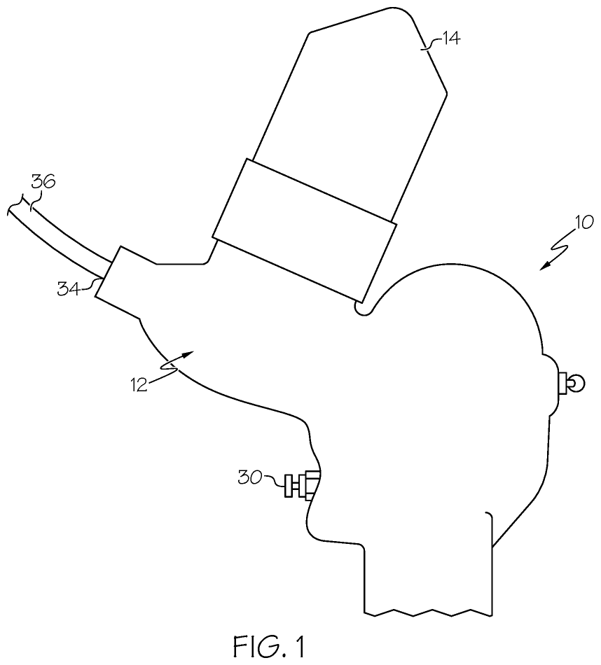

FIG. 1 shows a delivery system 10, which may be an agent (e.g., powder) delivery system. Delivery system 10 may include a handle body 12. Handle body 12 may include, or may be configured to receive, an enclosure 14 (or other source or container) storing a material (e.g., an agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. patent application Ser. No. 16/589,633, filed Oct. 1, 2019, published as U.S. Patent Application Publication No. 2020/0100986 A1 on Apr. 2, 2022, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source in fluid communication with delivery system 10. Actuation mechanism 30 may be selectively actuated (e.g., manually depressible) or otherwise moved or actuated to control delivery of a material (e.g., a powdered agent) and pressurized fluid. The pressurized fluid alone, or a combination of a powdered agent and the fluid, may be delivered from an outlet 34 of handle body 12. Outlet 34 may be in fluid communication with a catheter 36 or another delivery conduit for delivering the combination of agent and fluid to a desired location within a body lumen of a patient.

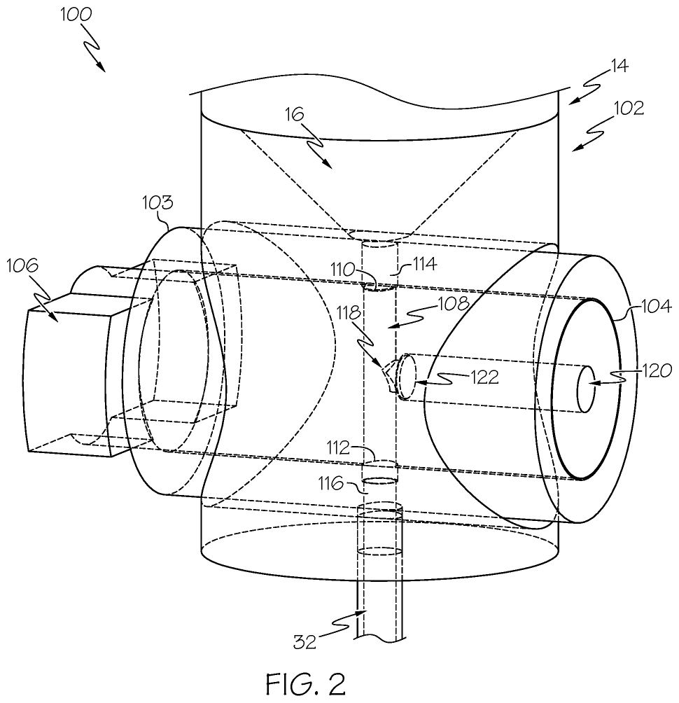

FIG. 2 shows aspects of an exemplary valve assembly 100. Valve assembly 100 may be at least partially housed within handle body 12 of delivery system 10. In some embodiments, valve assembly 100 may be selectively actuated (e.g., moved) by actuation mechanism 30. In other embodiments, valve assembly 100 may be actuated by a separate actuator, such as a control knob 106 (discussed in further detail below). In this instance, actuation mechanism 30 may be configured to deliver pressurized from the pressurized fluid source to valve assembly 100, and control knob 106 may be configured to actuate (e.g. move) one or more components of valve assembly 100 to selectively guide the pressurized fluid through valve assembly 100.

In further embodiments, actuation mechanism 30 may be configured to simultaneously establish fluid communication between the pressurized fluid source and valve assembly 100 and also actuate control knob 106 to control movement of the one or more components of valve assembly 100. In other words, an operator may interact only with actuation mechanism 30, and actuation mechanism 30 may, in turn, actuate control knob 106. Although not shown, actuation mechanism 30 and/or control knob 106 may include one or more other actuation elements, such as, for example, a button, a slider, a lever, a trigger, a dial, and various other suitable actuators. As described herein, actuation of control knob 106 may control delivery of pressurized fluid and agent through valve assembly 100.

Valve assembly 100 may include a housing 102, a fixed body 103 at least partially disposed within housing 102, a movable body 104 disposed within fixed body 103, and control knob 106 coupled to movable body 104 (e.g., to one end of movable body 104). Control knob 106 may extend outwardly from an opening on housing 102 to allow an operator to access and actuate control knob 106. Housing 102 may be attached to enclosure 14 (e.g., by any of the mechanisms discussed above with respect to enclosure 14 and handle body 12), and specifically a funnel 16 extending from enclosure 14. Funnel 16 may extend at least partially into housing 102, and may be configured to receive the agent stored in enclosure 14, such as via gravitational forces, to guide the agent into valve assembly 100. Funnel 16 may have a cone-shaped profile with a tapering sidewall that extends radially-inward toward a center opening of valve assembly 100 (e.g., an inlet 114).

Fixed body 103 may be coupled to housing 102, and specifically press fit through an opening of housing 102 downstream of enclosure 14 and funnel 16. Fixed body 103 may be fixed relative to housing 102, such that fixed body 103 is immovable relative to housing 102, enclosure 14, and funnel 16. As described in detail herein, fixed body 103 may include inlet 114 that is in fluid communication with enclosure 14 and funnel 16, and an outlet 116 that is in fluid communication with a tube 32 of delivery device 10. Movable body 104 may be coupled to fixed body 103, and specifically press fit through an opening of fixed body 103. Movable body 104 may be configured to move (e.g., rotate) within the opening and relative to fixed body 103, housing 102, enclosure 14, and funnel 16. In some embodiments, fixed body 103 may be omitted entirely such that movable body 104 may be coupled to housing 102, and housing 102 may include inlet 114 and outlet 116.

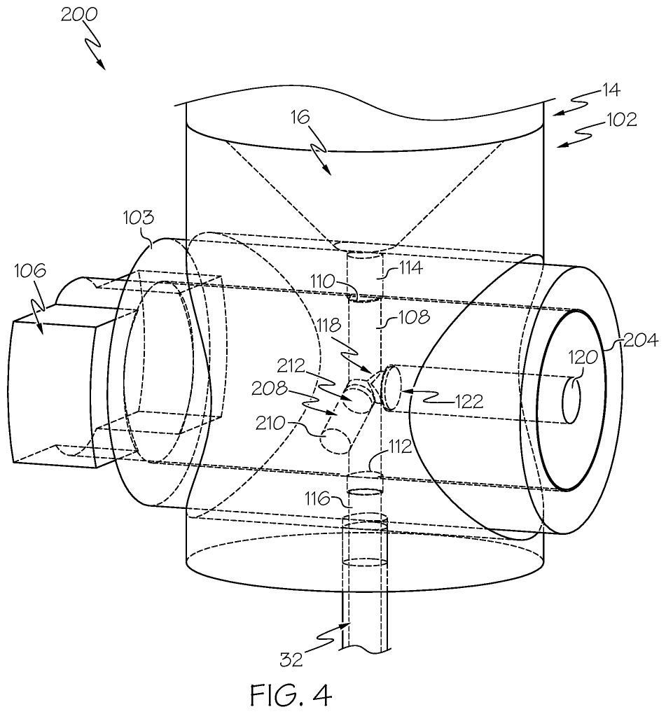

Still referring to FIG. 2, valve assembly 100 may include one or more internal channels within movable body 104. For example, valve assembly 100 may include a channel 108 extending through movable body 104, and channel 108 may have a longitudinal length defined between a first open end 110 and a second open end 112. In other embodiments, a valve assembly may include additional channels within a movable body (see FIG. 4). Valve assembly 100 may include inlet 114 along a first end of housing 102, and outlet 116 along a second end of fixed body 103. Inlet 114 may be in fluid communication with funnel 16, and in further fluid communication with channel 108 in configurations in which at least one of first open end 110 and/or second open end 112 is aligned thereto. Inlet 114 may be configured to guide a pressurized fluid from channel 108 into enclosure 14 for agitating an agent stored therein, and to guide the agitated agent out of enclosure 14 (e.g., through funnel 16) and into channel 108.

Outlet 116 may be in fluid communication with tube 32 of delivery device 10, and in further fluid communication with channel 108 in configurations in which at least one of first open end 110 and/or second open end 112 is aligned thereto. Outlet 116 may be configured to guide the agitated agent received in channel 108 (e.g., from enclosure 14) to tube 32. In some embodiments, tube 32 may include a hypotube that is fluidly coupled to catheter 36 (see FIG. 1). Tube 32 may be configured to guide a mixture of the agitated agent and pressurized fluid received from valve assembly 100 to catheter 36 for delivery to a patient.

Still referring to FIG. 2, valve assembly 100 may include a fluidics channel 120 extending through movable body 104 in a transverse (e.g., approximately perpendicular) orientation relative to channel 108. Fluidics channel 120 may be in fluid communication with channel 108 at a junction 118 that is positioned along an intermediate portion of channel 108 between first open end 110 and second open end 112. Fluidics channel 120 may be in further fluid communication with the pressurized fluid source, such that valve assembly 100 may be configured to receive the pressurized fluid via fluidics channel 120.

In the example, valve assembly 100 may include an insert 122 positioned within fluidics channel 120, such as adjacent to junction 118. Insert 122 may include a porous mesh that is configured to allow the pressurized fluid received from the pressurized fluid source to pass through fluidics channel 120 and into channel 108 at the junction 118, while inhibiting the agent received in channel 108 from entering fluidics channel 108.

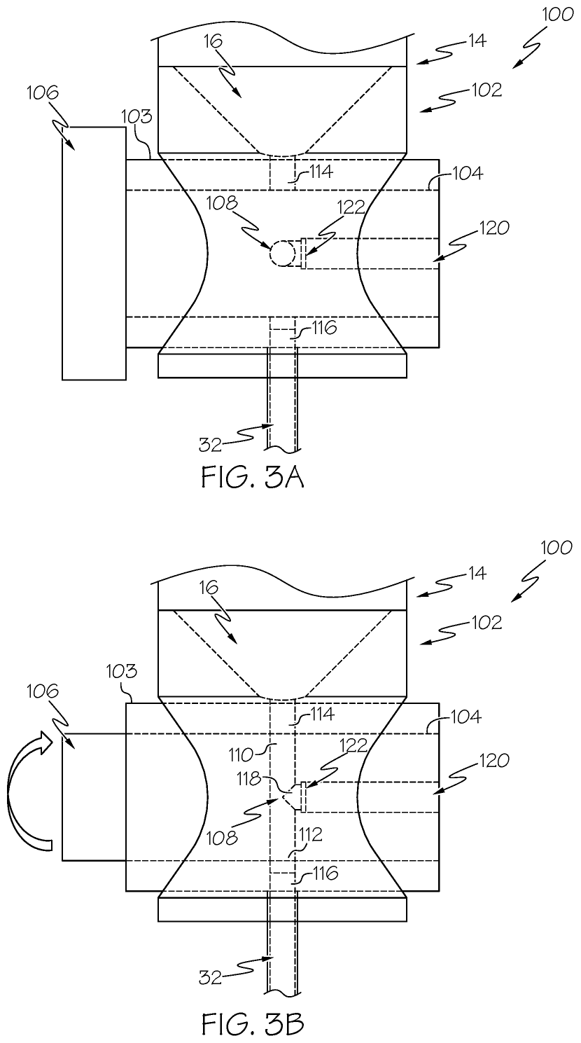

Valve assembly 100 may be configured to transition between a plurality of configurations, such as, for example, a non-delivery configuration (FIG. 3A) and a delivery configuration (FIG. 3B). As described herein, movable body 104 may be configured to move (e.g., rotate) relative to housing 102, fixed body 103, enclosure 14, funnel 16, and/or tube 32 in response to an actuation of control knob 106 between a plurality of positions corresponding to the configurations of valve assembly 100. In some embodiments, valve assembly 100 may include one or more visual indicators (e.g., markers, indicia, colors, etc.) and/or physical stops (e.g., protrusions, tabs, recesses, etc.) defining the plurality of positions of control knob 106 that correspond to the respective configurations of valve assembly 100 to facilitate an actuation of control knob 106 by a user.

For example, movable body 104 may be configured to rotate about an axis that is approximately coaxial with fluidics channel 120. It should be appreciated that channel 108 and/or fluidics channel 120 may be configured to move (e.g., rotate) simultaneously with movable body 104, thereby adjusting an alignment and/or orientation of the respective channels relative to enclosure 14, funnel 16, and/or tube 32.

In exemplary use, as seen in FIG. 3A, valve assembly 100 may be in the non-delivery configuration when control knob 106 is actuated to position movable body 104 in a first position in which channel 108 is misaligned with inlet 114 and outlet 116. Stated differently, movable body 104 may be moved (e.g., rotated) relative to housing 102, fixed body 103, enclosure 14, funnel 16, and/or tube 32 such that first open end 110 and second open end 112 are fluidly decoupled from inlet 114 and outlet 116. In this instance, the pressurized fluid received in channel 108 from fluidics channel 120 may be inhibited from entering enclosure 14 and funnel 16 via inlet 114, and/or tube 32 via outlet 116, while movable body 104 is in the first position. With first open end 110 and second open end 112 forming terminal/closed pathways, the pressurized fluid may be maintained within channel 108 and/or fluidics channel 120 when movable body 104 is in the first position. In other embodiments, the pressurized fluid may be inhibited from being delivered to valve assembly 100 while movable body 104 is in the first position.

It should be appreciated that channel 108 may be oriented at various suitable orientations and/or alignments relative to inlet 114 and outlet 116 when movable body 104 is in the first position, such that the corresponding position of channel 108 may include multiple positions in which first open end 110 and second open end 112 are misaligned with each of inlet 114 and outlet 116. Although channel 108 is shown in a particular alignment relative to inlet 114 and outlet 116 in FIG. 3A (e.g., an alignment in which a longitudinal axis of channel 108 is approximately 90 degrees offset from longitudinal axes of inlet 114 and outlet 116, such that the longitudinal axis of channel 108 extends into/out of the page of FIG. 3A), it should be understood that the first position of movable body 104 may include other possible orientations and/or alignments of channel 108 without departing from a scope of this disclosure.

Referring now to FIG. 3B, valve assembly 100 may be transitioned to the delivery configuration when control knob 106 is actuated to move (e.g., rotate) movable body 104 to a second position in which channel 108 is aligned with inlet 114 and outlet 116. Stated differently, movable body 104 may be rotated relative to housing 102, fixed body 103, enclosure 14, funnel 16, and/or tube 32 to fluidly couple first open end 110 and second open end 112 with inlet 114 and outlet 116, or vice versa. In this instance, the pressurized fluid received in channel 108 from fluidics channel 120 may enter enclosure 14 and funnel 16 via inlet 114, and/or tube 32 via outlet 116, when movable body 104 is in the second position.

With first open end 110 forming an open pathway towards inlet 114 when movable body 104 is in the second position, at least a first portion of the pressurized fluid received from fluidics channel 120 may be guided through channel 108 and into enclosure 14 and funnel 16 via inlet 114. The first portion of the pressurized fluid may move through funnel 16 and agitate the agent stored within funnel 16 and/or enclosure 14. Upon agitating and/or fluidizing the agent, a mixture of the agent and pressurized fluid may be guided through inlet 114 and into channel 108 prior to exiting channel 108 and entering tube 32 via outlet 116. With tube 32 in fluid communication with catheter 36, the mixture may be delivered to a patient via catheter 36.

Still referring to FIG. 3B, with second open end 112 forming an open pathway towards outlet 116 when movable body 104 is in the second position, at least a second portion of the pressurized fluid received from fluidics channel 120 may be guided through channel 108 and into tube 32 via outlet 116. It should be appreciated that the second portion of the pressurized fluid may be guided through channel 108 and toward outlet 116 as the first portion of the pressurized fluid is simultaneously guided through channel 108 and toward inlet 114. Insert 122 may be configured to inhibit any materials and/or agent maintained within enclosure 14, funnel 16, tube 32, and/or catheter 36 from flowing from channel 108 into fluidics channel 120.

In some embodiments, fluidics channel 120 may be configured to maintain fluid communication with the pressurized fluid source when movable body 104 is in the first and second positions. Stated differently, fluidics channel 120 may continue to receive the pressurized fluid source irrespective of a relative position of movable body 104. In other embodiments, fluidics channel 120 may remain in fluid communication with the pressurized fluid source as movable body 104 moves from the first position (FIG. 3A) to the second position (FIG. 3B). In the example, fluidics channel 120 may be configured to maintain a fixed orientation relative to enclosure 14, funnel 14, tube 32, and/or housing 102 as movable body 104 moves between the first and second positions.

Referring now to FIG. 4, aspects of another exemplary valve assembly 200 are shown. Valve assembly 200 may be configured similar to valve assembly 100 except for the differences explicitly described herein, such that like reference numerals are used to identify like components. Valve assembly 200 may be housed within handle body 12. Valve assembly 200 may include a movable body 204 that is at least partially housed within fixed body 103, and coupled to control knob 106. As discussed above with respect to movable body 104 of valve assembly 100, movable body 204 may be selectively actuated by actuation mechanism 30, which may interact with control knob 106 (see FIG. 1), or separately by control knob 106. In the embodiment, valve assembly 200 may include two or more internal channels within movable body 204.

For example, valve assembly 200 may include a first channel 108 (discussed above with respect to FIGS. 2-3B) and a second channel 208 extending through movable body 204. Second channel 208 may have a transverse orientation relative to first channel 108. For example, second channel 208 may be angled relative to first channel 108 between a range of about 50 degrees to about 70 degrees, such as 60 degrees. In the example, second channel 208 may be in fluid communication with first channel 108 and fluidics channel 120 at junction 118. Second channel 208 may have a longitudinal length defined between junction 118 and an open end 210. In the example, second channel 208 may have a longitudinal length that is less than the longitudinal length of first channel 108. In other embodiments, first channel 108 and second channel 208 may have substantially similar longitudinal lengths.

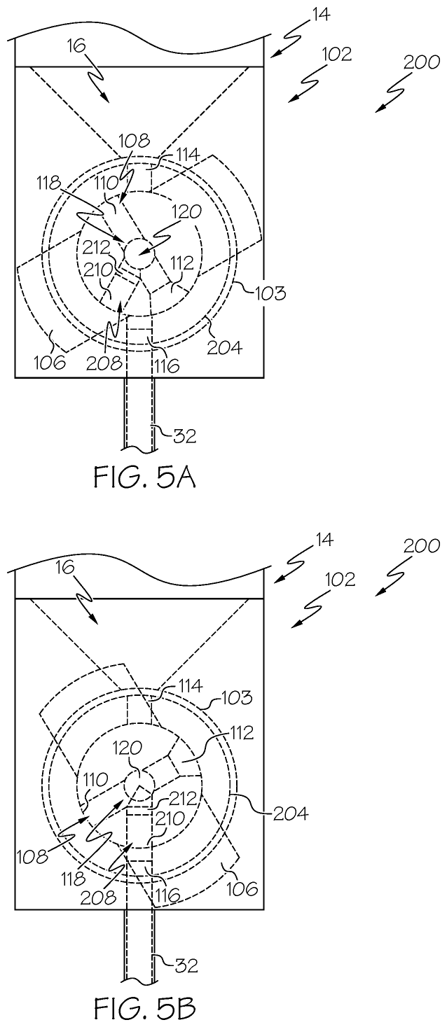

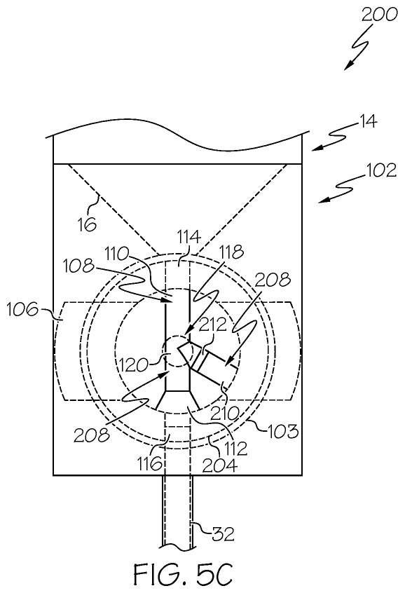

Still referring to FIG. 4, valve assembly 200 may include an insert 212 positioned within second channel 208, such as adjacent to the junction 118. Insert 212 may be substantially similar to insert 122 described above, such that insert 212 may be configured to allow the pressurized fluid received from the pressurized fluid source (via fluidics channel 120) to pass into second channel 208, while inhibiting the agent received in first channel 108 from entering second channel 208. As described herein, second channel 208 may be configured to deliver pressurized fluid to a patient in isolation from the agent when movable body 204 is moved to a second position (FIG. 5B) in which second channel 208 is in alignment with outlet 116.

In some embodiments, one or more of the open ends of first channel 108 may have a widened and/or expanded configuration to facilitate a flow of the pressurized fluid passing through first channel 108. For example, second open end 112 may have a widened configuration for enhancing the flow of pressurized fluid exiting first channel 108 via second open end 112. In this instance, the widened configuration of second open end 112 may permit a greater flow of fluid and/or agent entering outlet 116 for delivery through tube 32. In another example, first open end 110 may have a widened configuration for permitting a greater flow of fluid entering inlet 114 for agitating the agent stored in enclosure 14 and funnel 16. Although described with respect to valve assembly 200, valve assembly 100 may also include widened configurations of second open end 112 and/or first open end 110.

Valve assembly 200 may be configured to transition between a plurality of configurations, such as, for example, a non-delivery configuration (FIG. 5A), a fluid delivery configuration (FIG. 5B), and an agent delivery configuration (FIG. 5C). Movable body 204 may be configured to move (e.g., rotate) relative to housing 102, fixed body 103, enclosure 14, funnel 16, and/or tube 32 in response to an actuation of control knob 106 between a plurality of positions corresponding to the configurations of valve assembly 200. For example, movable body 204 may be configured to rotate about an axis that is coaxial with a central longitudinal axis of fluidics channel 120.

It should be appreciated that first channel 108, second channel 208, and fluidics channel 120 may be configured to move (e.g., rotate) simultaneously with movable body 204, thereby adjusting an alignment and/or orientation of the respective channels relative to enclosure 14, funnel 16, and/or tube 32. Although valve assembly 200 is described below with respect to a first, second, and third position of movable body 204, it should be understood that such terminology is merely exemplary such that valve assembly 200 may be shifted between the plurality of configurations in any respective sequence. By way of illustrative example only, valve assembly 200 may be transitions from the non-delivery configuration to the agent delivery configuration, the agent delivery configuration to the fluid delivery configuration, the fluid delivery configuration to the non-delivery configuration, or various other suitable sequences.

In exemplary use, as seen in FIG. 5A, valve assembly 200 may be in the non-delivery configuration when control knob 106 is actuated to move movable body 204 to a first position in which first channel 108 and second channel 208 are misaligned with inlet 114 and outlet 116, respectively. Stated differently, movable body 204 may be moved (e.g., rotated) relative to housing 102, fixed body 103, enclosure 14, funnel 16, and/or tube 32 such that first open end 110, second open end 112, and open end 210 are fluidly decoupled from inlet 114 and outlet 116. In this instance, the pressurized fluid received in first channel 108 and second channel 208 from fluidics channel 120 may be inhibited from entering enclosure 14 via inlet 114, and/or tube 32 via outlet 116, when movable body 204 is in the first position. With first open end 110, second open end 112, and open end 210 forming terminal/closed pathways, the pressurized fluid may be maintained within first channel 108, second channel 208, and/or fluidics channel 120 when movable body 204 is in the first position.

It should be appreciated that each channel 108, 208 may be oriented at various suitable orientations and/or alignments relative to inlet 114 and outlet 116 when movable body 204 is in the first position, such that the corresponding positions of channels 108, 208 may include multiple positions in which first open end 110, second open end 112, and open end 210 are each misaligned with inlet 114 and outlet 116. Accordingly, although the channels 108, 208 are shown in a particular alignment relative to inlet 114 and outlet 116 in FIG. 5A, it should be understood that the first position of the movable body 204 may include other possible orientations and/or alignments of channels 108, 208.

Referring now to FIG. 5B, valve assembly 200 may be transitioned to the fluid delivery configuration when control knob 106 is actuated to move (e.g., rotate) movable body 204 to a second position in which second channel 208 is aligned with outlet 116, and first channel 108 remains misaligned with each of inlet 114 and outlet 116. Stated differently, movable body 204 may be rotated relative to housing 102, fixed body 103, enclosure 14, funnel 16, and/or tube 32 to fluidly couple open end 210 with outlet 116, and maintain first open end 110 and second open end 112 fluidly decoupled from inlet 114 and outlet 116. Pressurized fluid from the pressurized fluid source may flow into fluidics channel 120, through insert 122 and junction 118, and into second channel 208. In this instance, the pressurized fluid received in second channel 208 from fluidics channel 120 may enter tube 32 via outlet 116 when movable body 204 is in the second position.

In this instance, valve assembly 200 may be configured to deliver the pressurized fluid through tube 32 and into catheter 36 without the agent. When in the fluid delivery configuration of FIG. 5B, valve assembly 200 may clear any existing materials and/or agent maintained within tube 32 and/or catheter 36 by dispensing the pressurized fluid therethrough via second channel 208 without introducing any additional materials (e.g., the agent). Insert 212 may be configured to inhibit any of the existing materials and/or agent maintained within tube 32 and/or catheter 36 from flowing through second channel 208 and into first channel 108 and/or fluidics channel 120.

Referring now to FIG. 5C, valve assembly 200 may be transitioned to the agent delivery configuration when control knob 106 is actuated to move (e.g., rotate) movable body 204 to a third position in which first channel 108 is aligned with inlet 114 and outlet 116, and second channel 208 is misaligned with inlet 114 and outlet 116. Stated differently, movable body 204 may be rotated relative to housing 102, fixed body 103, enclosure 14, funnel 16, and/or tube 32 to fluidly couple first open end 110 and second open end 112 with inlet 114 and outlet 116, or vice versa. Accordingly, open end 210 may be fluidly decoupled from outlet 116. In this instance, the pressurized fluid received in first channel 108 from fluidics channel 120 may enter enclosure 14 via inlet 114, and/or tube 32 via outlet 116, when movable body 204 is in the third position.

For example, with first open end 110 forming an open pathway towards inlet 114 when movable body 204 is in the third position, at least a first portion of the pressurized fluid received from fluidics channel 120 may be guided through first channel 108 and into enclosure 14 via inlet 114. The first portion of the pressurized fluid may move through funnel 16 and agitate the agent within funnel 16 and/or enclosure 14. Upon agitating the agent, a mixture of the agent and pressurized fluid may be guided through inlet 114, first channel 108, and outlet 116 prior to entering tube 32.

Still referring to FIG. 5C, with second open end 112 forming an open pathway towards outlet 116 when movable body 204 is in the third position, at least a second portion of the pressurized fluid received from fluidics channel 120 may be guided through first channel 108 and into tube 32 via outlet 116. It should be appreciated that the second portion of the pressurized fluid may be guided through first channel 108 and toward outlet 116 as the first portion of the pressurized fluid is simultaneously guided through first channel 108 and toward inlet 114.

Although not shown, it should be appreciated that valve assembly 200 may be further transitioned to an agitated configuration in which movable body 204 may be moved (e.g., rotated) to a fourth position with open end 210 aligned with inlet 114. In this instance, second channel 208 may be fluidly coupled to enclosure 14 and funnel 16 such that the pressurized fluid received from fluidics channel 120 may be guided toward the agent stored therein. In this instance, valve assembly 200 may be configured to agitate the agent within enclosure 14 and/or funnel 16 without delivering any portion of the agent to tube 32.

Although each of valve assembly 100 and valve assembly 200 are shown and described herein as having bodies 104, 204 that are movable relative to a respective housing 102, it should be appreciated that in other embodiments the bodies 104, 204 (and particularly the one or more channels) may remain relatively fixed. In this instance, enclosure 14 and/or funnel 16 may be selectively moved to align inlet 114 with said channels.

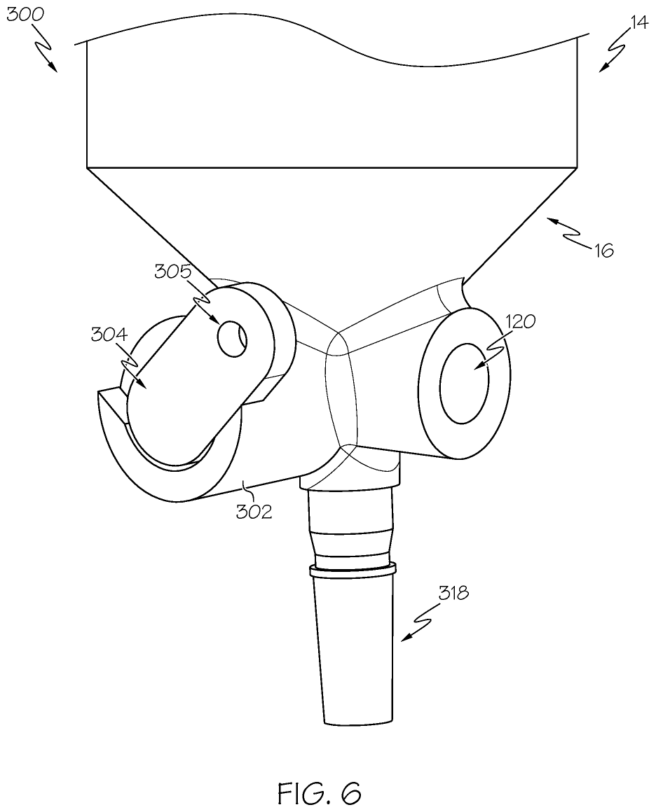

Referring now to FIG. 6, aspects of another exemplary valve assembly 300 are shown. Valve assembly 300 may be configured similar to valve assembly 100 except for the differences explicitly described herein, such that like reference numerals are used to identify like components. Valve assembly 300 may be housed within handle body 12. Valve assembly 300 may include a housing 302, an exit port 318, and fluidics channel 120. Housing 302 may be coupled to enclosure 14, and particularly to funnel 16.

Valve assembly 300 may further include a movable body 304 that is at least partially housed within housing 302. At least a portion of movable body 304 may include an opening 305 to facilitate connection to an actuator (not shown). For example, a linking mechanism (e.g., a rod, a wire, a cable, etc.) may be coupled to the actuator and to movable body 304 at opening 305. Accordingly, movable body 304 may be configured to move (e.g., pivot, rotate, translate, etc.) relative to housing 302 in response to actuation of the actuator. It should be appreciated that the actuator may be separate from actuation mechanism 30 (FIG. 1), such that valve assembly 300 may be configured to actuate movable body 304 (via the actuator) and deliver the pressurized fluid (via actuation mechanism 30) independently of one another. In other embodiments, valve assembly 300 may be selectively actuated by actuation mechanism 30 (see FIG. 1). As described herein, movable body 304 may include one or more internal channels.

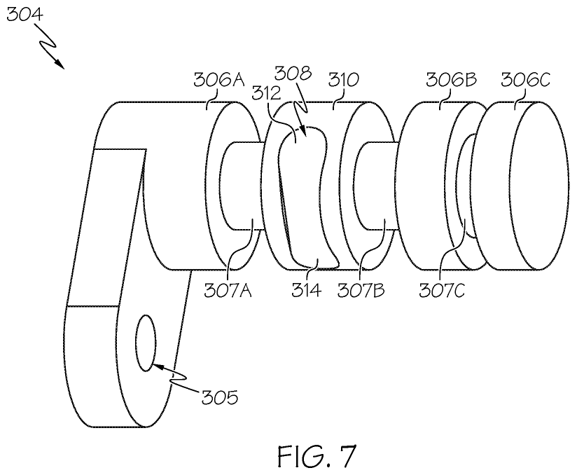

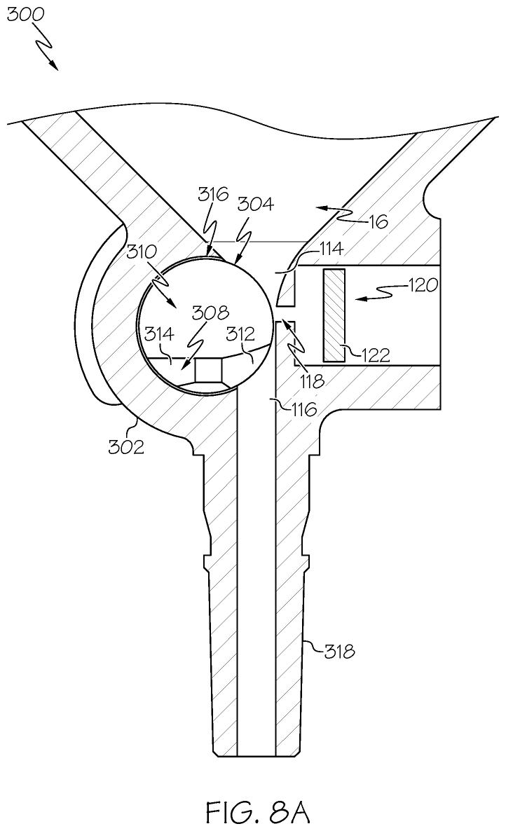

As seen in FIG. 7, movable body 304 may include a proximal portion 306A, a distal portion 306B, and a distal end 306C. Movable body 304 may further include an intermediate body 310 positioned between proximal portion 306A and distal portion 306B. Intermediate body 310 may have a channel 308 formed along a sidewall of intermediate body 310, such that channel 308 may be open along an exterior surface of intermediate body 310. A longitudinal length of channel 308 may be defined between a first open end 312 and a second open end 314. As described herein, channel 308 may be configured to receive an agent and/or pressurized fluid when movable body 304 is moved to one or more positions within housing 302.

Proximal portion 306A may be separated from intermediate body 310 by a first slot 307A, intermediate body 310 may be separated from distal portion 306B by a second slot 307B, and distal portion 306B may be separated from distal end 306C by a third slot 307C. Each of slots 307A, 307B, 307C may be sized and shaped to receive a corresponding mechanism therein. For example, each of first slot 307A and second slot 307B may be configured to receive a sealing mechanism, such as, for example, an O-ring. The sealing mechanisms (not shown) may be disposed about intermediate body 310 to fluidly seal channel 308 within housing 302. Third slot 307C may be configured to receive a fastening mechanism, such as, for example, a retaining clip. The fastening mechanism (not shown) may be configured to secure and maintain movable body 304 within housing 302 during movement (e.g., rotation) of movable body 304 relative to housing 302.

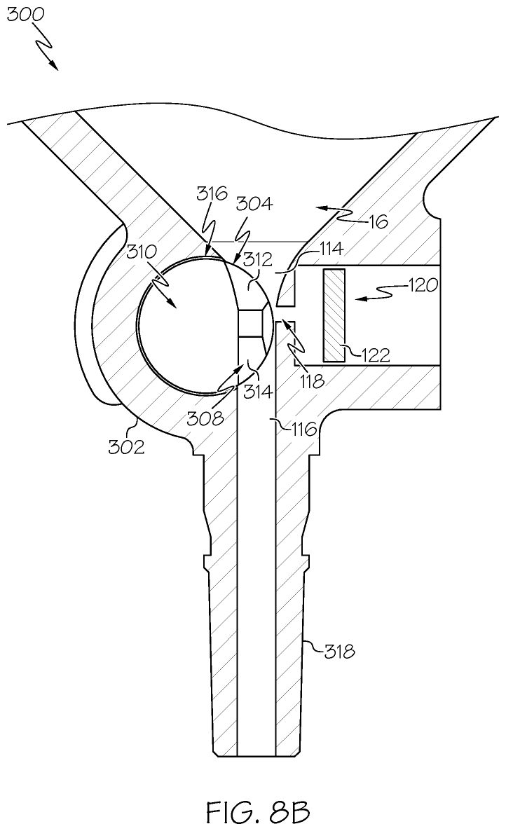

Valve assembly 300 may be configured to transition between a plurality of configurations, such as, for example, a non-delivery configuration (FIG. 8A), a fluid delivery configuration (also shown in FIG. 8A), and an agent delivery configuration (FIG. 8B). As described herein, movable body 304 may be configured to move (e.g., rotate) relative to housing 302, enclosure 14, funnel 16, and/or exit port 318 in response to an actuation of an actuator (not shown) between a plurality of positions corresponding to the configurations of valve assembly 300. It should be appreciated that intermediate body 310 and channel 308 may be configured to move (e.g., rotate) simultaneously with movable body 304, thereby adjusting an alignment and/or orientation of channel 308 relative to housing 302, enclosure 14, and/or funnel 16. Further, valve assembly 300 may be configured to receive pressurized fluid from the pressurized fluid source in response to an actuation of actuation mechanism 30.

Referring to FIG. 8A, movable body 304 may be disposed within housing 302 with intermediate body 310 positioned adjacent to junction 118. Accordingly, any pressurized fluid received within housing 302 via fluidics channel 120 may encounter intermediate body 310 upon passing through insert 122 and junction 118. In exemplary use, valve assembly 300 may be in the non-delivery configuration when movable body 304 is in a first position, in which channel 308 is misaligned with inlet 114 and outlet 116, and the pressurized fluid is not actively delivered to fluidics channel 120 (from the pressurized fluid source in response to actuation of actuation mechanism 30). In this configuration, as discussed in detail below, the agent stored in enclosure 14 and funnel 16 may be inhibited from moving between inlet 114 and outlet 116 (e.g., via gravitational forces) with intermediate body 310 positioned therebetween to block a pathway of the agent.

Valve assembly 300 may be transitioned from the non-delivery configuration to the fluid delivery configuration upon maintaining movable body 304 in the first position, as shown in FIG. 8A, and actuating actuation mechanism 30 to deliver the pressurized fluid from the pressurized fluid source to fluidics channel 120. In this instance, channel 308 may remain misaligned with inlet 114 and outlet 116, thereby inhibiting the agent from exiting funnel 16. In the example, intermediate body 310 may be sized and/or shaped such that a clearance gap 316 is formed between an interior surface of housing 302 and an exterior surface of intermediate body 310.

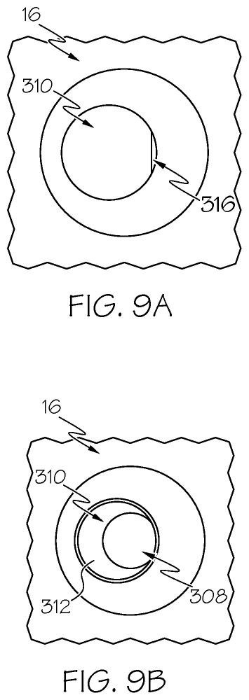

As best seen in FIG. 9A, the clearance gap 316 may be sized such that only the pressurized fluid may be received therein, thereby inhibiting any material (e.g., the agent) from passing therethrough. In other words, FIG. 9A shows a partial perspective view from within funnel 16 in which movable body 304 may be positioned in the first position with intermediate body 310 covering the opening of funnel 16. Clearance gap 316 may be formed between intermediate body 310 and an inner surface of housing 302, thereby allowing pressurized fluid to pass through clearance gap 316 and into funnel 16.

Accordingly, with movable body 304 in the first position, valve assembly 300 may be configured to permit the pressurized fluid to at least partially bypass movable body 304 via the clearance gap 316. In other words, when valve assembly 300 is in the fluid delivery configuration, the pressurized fluid received at junction 118 from fluidics channel 120 may pass around intermediate body 310. In this instance, at least a first portion of the pressurized fluid may be guided toward inlet 114 and at least a second portion of the pressurized fluid may be guided toward outlet 116 via the clearance gap 316 formed between intermediate body 310 and housing 302.

The first portion of the pressurized fluid may move through funnel 16 and agitate the agent stored within funnel 16 and/or enclosure 14. Due to the size of clearance gap 316 relative to a particulate size of the agent, movable body 304 may be configured to inhibit the agitated agent in enclosure 14 and funnel 16 from exiting while movable body 304 remains in the first position. Accordingly, a mixture of the agent and the pressurized fluid may be inhibited from moving toward outlet 116 and exit port 318. By allowing the first portion of the pressurized fluid to move through housing 302 while in the fluid delivery configuration, valve assembly 300 may be operable to prevent long-term pressurization of enclosure 14.

Still referring to FIG. 8A, valve assembly 300 may further permit delivery of at least the second portion of the pressurized fluid toward exit port 318 via outlet 116 without delivering any portion of the agent stored in enclosure 14. With exit port 318 in fluid communication with catheter 36, valve assembly 300 may be configured to clear any existing materials and/or agent maintained within exit port 318 and/or catheter 36, such as from a prior delivery, by only permitting delivery of the pressurized fluid while in the fluid delivery configuration.

It should be appreciated that channel 308 may be oriented at various suitable orientations and/or alignments relative to inlet 114 and outlet 116 when movable body 304 is in the first position. Although channel 308 is shown in a particular alignment relative to inlet 114 and outlet 116 in FIG. 8A, it should be understood that the first position of movable body 304 may include other possible orientations and/or alignments of channel 308.

Referring now to FIG. 8B, valve assembly 300 may be transitioned to the agent delivery configuration when the actuator is actuated to move (e.g., rotate) movable body 304 to a second position in which channel 308 is aligned with inlet 114 and outlet 116. Stated differently, movable body 304 may be rotated relative to housing 302, enclosure 14, funnel 16, and/or exit port 318 to fluidly couple first open end 312 and second open end 314 with inlet 114 and outlet 116, or vice versa. In this instance, the pressurized fluid received in enclosure 14 and/or funnel 16 via inlet 114 may guide the agitated agent into channel 308 at first open end 312, as seen in FIG. 9B. In other words, FIG. 9B shows a partial perspective view from within funnel 16 in which movable body 304 may be positioned in the second position with first open end 312 in alignment with the opening of funnel 16. Channel 308 may be in fluid communication with funnel 16, thereby allowing the pressurized fluid to pass into funnel 16, and subsequently allowing a mixture of the agitated agent and fluid to enter channel 308 from funnel 16 via first open end 312.

With channel 308 forming an open pathway between inlet 114 and outlet 116 when movable body 304 is in the second position, the agitated agent and pressurized fluid received in enclosure 14 may move through funnel 16 and into channel 308 prior to exiting intermediate body 310 at outlet 116. As described above, exit port 318 may be in fluid communication with catheter 36 such that the mixture of the agent and pressurized fluid received in exit port 318 may be delivered to a patient via catheter 36 (FIG. 1). Insert 122 may be configured to inhibit the agent moving through channel 308 from flowing into fluidics channel 120.

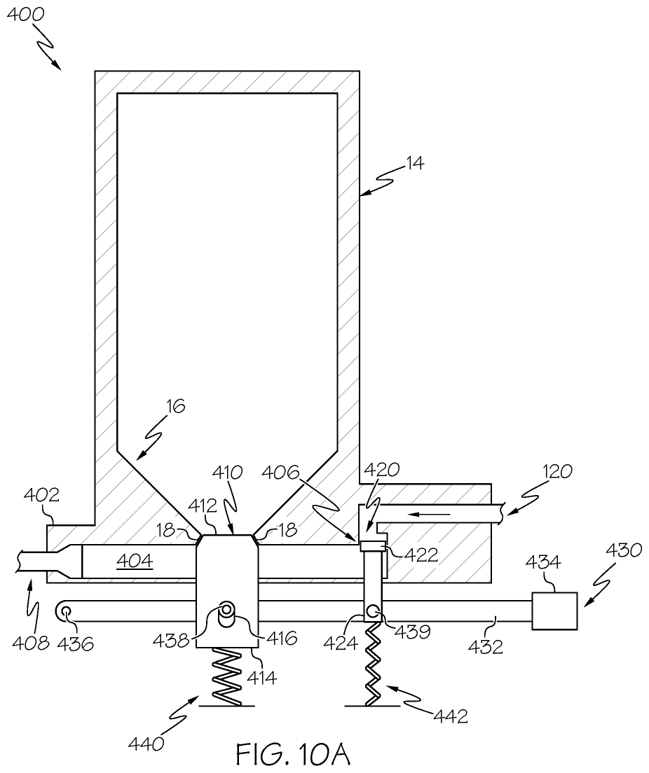

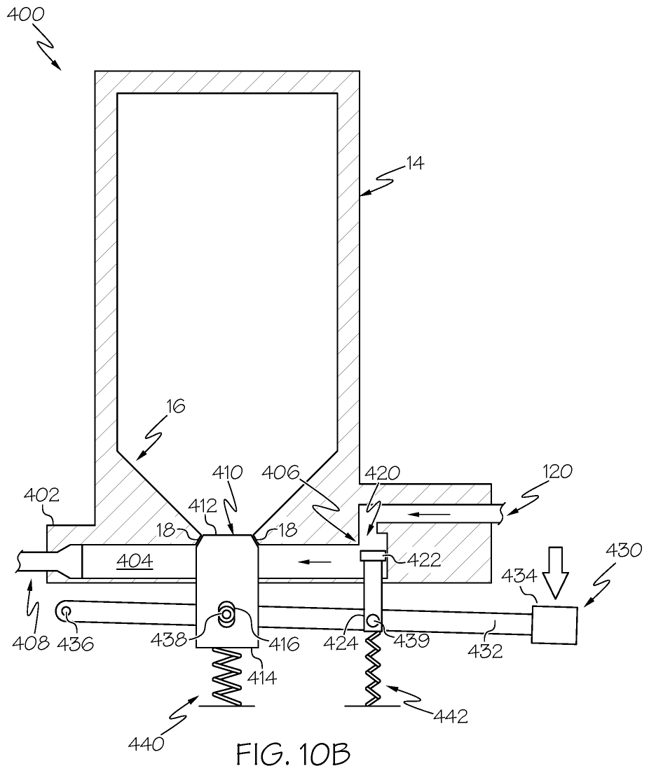

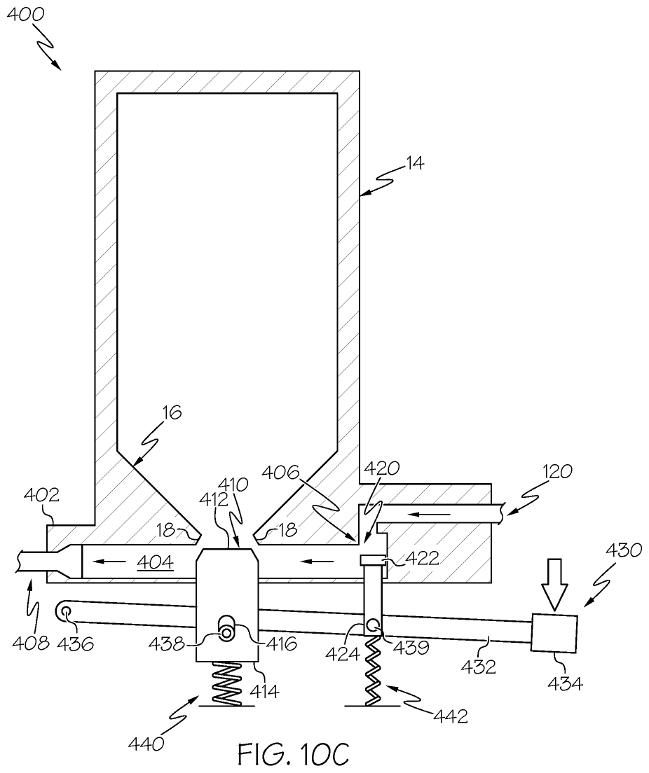

Referring now to FIGS. 10A-10C, aspects of another exemplary valve assembly 400 are shown. Valve assembly 400 may be configured similar to valve assembly 100 except for the differences explicitly described herein, such that like reference numerals are used to identify like components. Valve assembly 400 may be housed within handle body 12 (FIG. 1), and may include a housing 402 that is coupled to enclosure 14 via funnel 16. In some embodiments, housing 402 may be integral with funnel 16 and enclosure 14, while in other embodiments housing 402 may be selectively attached thereto. In the embodiment, valve assembly 400 may include one or more internal channels.

Referring specifically to FIG. 10A, valve assembly 400 may include a channel 404 extending through housing 402. Channel 404 may have a longitudinal length defined between an inlet 406 and an outlet 408. In the example, inlet 406 may be fluidly coupled to fluidics channel 120 for receiving the pressurized fluid in channel 404, and outlet 408 may be fluidly coupled to catheter 36 (see FIG. 1) for delivering an agent received in channel 404 (from enclosure 14) to a patient. Valve assembly 400 may include one or more movable bodies for selectively controlling a delivery of the pressurized fluid and agent to the patient.

For example, valve assembly 400 may include at least a first movable body 410 and a second movable body 420, and each of the movable bodies 410, 420 may be at least partially received within channel 404. First movable body 410 may include a first end 412 and a second end 414, with first end 412 positioned within channel 404 and adjacent to a center opening of funnel 16 in at least some configurations. In some embodiments, the center opening of funnel 16 may be at least partially defined by one or more surfaces 18. The surfaces 18 may at least partially define a junction between funnel 16 and channel 404. In the example, surfaces 18 may be at least partially angled to taper outwardly from a center opening of funnel 16.

Still referring to FIG. 10A, first end 412 may be sized, shaped, and otherwise configured to interface with the surfaces 18 to thereby seal the center opening of funnel 16 when first movable body 410 is in a first position in which first end 412 abuts against the surfaces 18. At least some surfaces of first end 412 may have a complementary shape to a shape of surfaces 18. For example, first end 412 may include surfaces and/or edges that taper inwardly to mate with surfaces 18. Second end 414 may be positioned outside of channel 404, and first movable body 410 may be coupled to a biasing mechanism 440 (e.g., a spring) at second end 414. Biasing mechanism 440 may be configured to urge first movable body 410 to the first position, such that first end 412 may be biased toward the surfaces 18 of funnel 16. As described herein, valve assembly 400 may include an actuator 430 (e.g., a lever assembly) configured to move first movable body 410 between one or more positions relative to funnel 16.

Second movable body 420 may include a first end 422 and a second end 424, with first end 422 positioned within channel 404 and adjacent to inlet 406. In the example, first end 422 may be sized, shaped, and otherwise configured to interface with inlet 406 to thereby seal channel 404 from fluidics channel 120 when second movable body 420 is in a first position in which first end 422 abuts against one or more surfaces defining inlet 406.

Still referring to FIG. 10A, second end 424 may be positioned outside of channel 404, and second movable body 420 may be coupled to a biasing mechanism 442 (e.g., a spring) at second end 424. Biasing mechanism 442 may be configured to urge second movable body 420 to the first position, such that first end 422 may be biased towards the surfaces defining inlet 406. As described herein, actuator 430 may be configured to move second movable body 420 between one or more positions relative to inlet 406.

Actuator 430 may be a lever assembly that includes a rod/shaft 432 having a handle 434 at one end, and a pivot joint 436 at an opposite end. Handle 434 may be configured to move (e.g., pivot) shaft 432 between one or more positions about pivot joint 436. In some embodiments, actuator 430 may be manually manipulated via handle 434. In other embodiments, actuator 430 may be automatically actuated in response to an actuation of actuation mechanism 30 (FIG. 1). In further embodiments, actuation mechanism 30 may be omitted entirely such that actuator 430 may be configured to release the pressurized fluid from the pressurized fluid source and simultaneously move movable bodies 410, 420.

Still referring to FIG. 10A, actuator 430 may be coupled to each of first movable body 410 and second movable body 420 via at least one engagement mechanism. For example, first movable body 410 may be coupled to actuator 430, and particularly shaft 432, via a pin 438 received within a slot 416 formed in first movable body 410. Second movable body 420 may be coupled to shaft 432 via a pin 439 received in an opening formed at second end 424. Accordingly, movement of shaft 432 (via actuation of handle 434) may provide for a corresponding movement of first movable body 410 and second movable body 420 between one or more positions.

Valve assembly 400 may be configured to transition between a plurality of configurations, such as, for example, a non-delivery configuration (FIG. 10A), a fluid delivery configuration (FIG. 10B), and an agent delivery configuration (FIG. 10C). As described herein, movable bodies 410, 420 may be configured to move (e.g., translate) relative to housing 402, enclosure 14, funnel 14, and/or channel 404 in response to an actuation of actuator 430 between a plurality of positions corresponding to the configurations of valve assembly 400.

In exemplary use, as seen in FIG. 10A, valve assembly 400 may be biased to the non-delivery configuration in which movable bodies 410, 420 are urged to the first position. In the first position, first movable body 410 may be engaged against surfaces 18 of funnel 16, and second movable body 420 may be engaged against inlet 406. Accordingly, valve assembly 400 may be configured to inhibit release of the agent from enclosure 14 and/or funnel 16 into channel 404, with first movable body 410 forming a fluid seal against surfaces 18. Valve assembly 400 may be further configured to inhibit release of the pressurized fluid from fluidics channel 120 into channel 404, with second movable body 420 forming a fluid seal at inlet 406.

Valve assembly 400 may be maintained in the non-delivery configuration absent applying an opposing (downward) force against biasing mechanisms 440, 442 (e.g., in response to an actuation of actuator 430). As seen in FIG. 10B, valve assembly 400 may be transitioned from the non-delivery configuration (FIG. 10A) to the fluid delivery configuration upon actuating handle 434 to at least partially compress biasing mechanisms 440, 442 to a first extent. First movable body 410 may remain in the first position relative to channel 404, in which first end 412 is positioned against surfaces 18, such that first movable body 410 remains coupled to funnel 16. Accordingly, the agent stored in enclosure 14 and funnel 16 may remain sealed therein by first movable body 410.

In response to moving handle 434, biasing mechanism 440 may be at least partially compressed to thereby move pin 438 within slot 416, such as from a first position (seen in FIG. 10A) to a second position (seen in FIG. 10B). Second movable body 420 may move to a second position relative to channel 404, in which first end 422 moves away from inlet 406 in response to a compression of biasing mechanism 442. In this instance, first end 422 may disengage the surfaces defining inlet 406, thereby decoupling second movable body 420 from inlet 406. Accordingly, the pressurized fluid in fluidics channel 120 may be received within channel 404. With second movable body 420 in the second position, the pressurized fluid may be received within channel 404 via inlet 406, thereby delivering the pressurized fluid toward outlet 408.

In some embodiments, the pressurized fluid may travel through channel 404 and exit outlet 408 when valve assembly 400 is in the fluid delivery configuration. For example, the pressurized fluid may travel around first movable body 410 and toward outlet 408. In other embodiments, the pressurized fluid may be received within channel 404 and inhibited from exiting outlet 408 by first movable body 410. In this instance, the pressurized fluid may be maintained within channel 404 and guided toward outlet 408 only when valve assembly 400 transitions to the agent delivery configuration (see FIG. 10C). By guiding the pressurized fluid into channel 404 prior to releasing the agent into channel 404 from enclosure 14 and funnel 16, valve assembly 400 may be configured to inhibit channel 404 from clogging, such as in instances when the agent is received in channel 404 prior to the pressurized fluid.

As seen in FIG. 10C, valve assembly 400 may be transitioned from the fluid delivery configuration (FIG. 10B) to the agent delivery configuration upon actuating handle 434 to further compress biasing mechanisms 440, 442 to a second extent that is greater than the first extent (FIG. 10B). First movable body 410 may move to a second position relative to channel 404, in which first end 412 moves away from funnel 16 in response to a compression of biasing mechanism 440 and movement of pin 438 within slot 416, such as from the second position (seen in FIG. 10B) to a third position (seen in FIG. 10C). In this instance, first end 412 may disengage the surfaces 18 defining the center opening of funnel 16, thereby decoupling first movable body 410 from funnel 16. Accordingly, the agent stored in enclosure 14 and funnel 16 may be received within channel 404.

Second movable body 420 may move to a third position relative to channel 404, in which first end 422 moves further away from inlet 406 (relative to the second position in FIG. 10B) in response to a greater compression of biasing mechanism 442. In this instance, first end 422 may remain disengaged from the surfaces defining inlet 406, thereby maintaining second movable body 420 in a decoupled state from inlet 406. Accordingly, the pressurized fluid in fluidics channel 120 may continue to be received within channel 404. In some embodiments, the second position of second movable body 404 may be substantially the same as the third position. With first movable body 410 in the second position and second movable body 420 in the third position, the pressurized fluid may be received within channel 404 via inlet 406 and the agent stored within enclosure 14 and/or funnel 16 may be received within channel 404 via the center opening of funnel 16. In this instance, the pressurized fluid may agitate the agent within channel 404. It should be appreciated that the pressurized fluid may be received within channel 404 prior to the release of the agent into channel 404 from enclosure 14 and/or funnel 16.

In some embodiments, at least a portion of the pressurized fluid may be guided into funnel 16 and/or enclosure 14, thereby agitating the agent therein. With first movable body 410 at least partially positioned within channel 404 between inlet 406 and outlet 408, the pressurized fluid may be directed around first movable body 410 by surfaces of first movable body 410, such that at least a portion of the pressurized fluid may be received within funnel 16 and/or enclosure 14.

Still referring to FIG. 10C, the pressurized fluid may agitate the agent in either channel 404 and/or in funnel 16 and enclosure 14 prior to guiding the agitated agent toward outlet 408 for delivery to catheter 36 (FIG. 1). Although a single actuator 430 is shown and described herein, it should be appreciated that valve assembly 400 may include a respective actuator for selectively controlling movement of each movable body 410, 420. Alternatively, actuator 430 may be configured to selectively move each of first movable body 410 and second movable body 420 independently of one another. As described above, in some embodiments actuator 430 may be coupled to actuation mechanism 30 such that actuator 430 may be configured to move in response to actuation of actuation mechanism 30, or vice versa. Accordingly, movable bodies 410, 420 may move between the one or more positions simultaneously with the release of the pressurized fluid from the pressurized fluid source by actuator 30.

In some embodiments, fluidics channel 120 may be configured to maintain fluid communication with the pressurized fluid source when movable bodies 410, 420 are in the respective positions. Stated differently, fluidics channel 120 may continue to receive the pressurized fluid source irrespective of a relative position of movable bodies 410, 420. In other embodiments, fluidics channel 120 may be in fluid communication with the pressurized fluid source in response to second movable body 420 moving from the first position (FIG. 10A) to the second position (FIG. 10B). In this instance, actuation of actuator 430 may provide for a corresponding release of pressurized fluid from the pressurized fluid source.

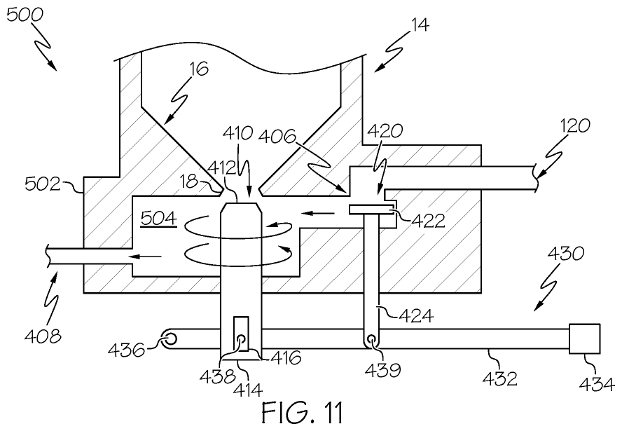

Referring now to FIG. 11, aspects of another exemplary valve assembly 500 are shown. Valve assembly 500 may be configured similar to valve assembly 400 except for the differences explicitly described herein, such that like reference numerals are used to identify like components. Valve assembly 500 may be housed within handle body 12 (FIG. 1), and may include a housing 502 that is coupled to enclosure 14 via funnel 16. In some embodiments, housing 502 may be integral with funnel 16 and enclosure 14, while in other embodiments housing 502 may be selectively attached thereto. In the embodiment, valve assembly 500 may include one or more internal channels.

For example, valve assembly 500 may include a channel 504 that has a greater cross-sectional profile relative to channel 404 of valve assembly 400 (FIGS. 10A-10B) and/or fluidics channel 120. In this instance, valve assembly 500 may be configured to generate a vortex within channel 504 when transitioning from the non-delivery configuration to the delivery configuration (FIG. 11). Stated differently, when movable bodies 410, 420 are moved to the respective second positions shown in FIG. 11, the agent stored in enclosure 14 and/or funnel 16 may enter channel 504 as the pressurized fluid from fluidics channel 120 is received at inlet 406. In this instance, the pressurized fluid may form a swirling vortex within channel 504 and around first movable body 410, as shown by the arrows in FIG. 11, to agitate the agent prior to delivery toward outlet 408.

In some embodiments, one or more surface features may be positioned within channel 504 for causing turbulence and enhancing a mixture and fluidization of the agent prior to delivery toward outlet 408. For example, valve assembly 500 may include one or more baffles, protrusions, dimples, recesses, cavities, and/or other suitable structures for forming a physical obstruction within channel 504. Such surface features may similarly be positioned within the respective channels of the valve assemblies 100, 200, 300, 400 shown and described above. It should be appreciated that, but for the differences described above, an exemplary use of valve assembly 500 may be substantially similar to valve assembly 400 shown and described above.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A valve assembly for a medical device, comprising:
an inlet that is in fluid communication with an enclosure of the medical device, wherein the enclosure stores an agent;
an outlet that is in fluid communication with a delivery conduit of the medical device; and
a body having a channel that is in fluid communication with a source of fluid, wherein the body is configured to move relative to the inlet and the outlet to selectively fluidly couple the channel with the enclosure and the delivery conduit;
wherein, in a first position of the body, the channel is misaligned with at least one of the inlet or the outlet, such that the delivery conduit is not in fluid communication with at least one of the enclosure or the source of fluid; and
wherein, in a second position of the body, the channel is aligned with the inlet and the outlet such that the delivery conduit is in fluid communication with the enclosure and the source of fluid.

2. The valve assembly of claim 1, wherein, in the first position, the channel is positioned in a transverse alignment relative to an axis extending between the inlet and the outlet.

3. The valve assembly of claim 1, wherein, in the second position, the channel is positioned in a parallel alignment relative to an axis extending between the inlet and the outlet.

4. The valve assembly of claim 1, wherein the channel is in fluid communication with the source of fluid when the body is in the first position and the second position.

5. The valve assembly of claim 1, wherein the body is configured to guide the fluid through the channel and into the enclosure via the inlet to agitate the agent within the enclosure when in the first position.

6. The valve assembly of claim 5, wherein the body is configured to guide a mixture of the fluid and the agent from the enclosure into the channel via the inlet, and the channel is configured to guide the mixture into the delivery conduit via the outlet.

7. The valve assembly of claim 1, wherein the body includes an insert positioned within the channel, the insert including a porous mesh.

8. The valve assembly of claim 7, wherein the insert is configured to inhibit the agent from moving through the insert towards the source of fluid, and to permit the fluid to pass through the insert.

9. The valve assembly of claim 1, wherein the channel is a first channel, and wherein the body includes a second channel that is in fluid communication with the first channel, the second channel having a length that is less than a length of the first channel.

10. The valve assembly of claim 9, wherein, in the first position and the second position of the body, the second channel is:
misaligned with the outlet;
in fluid communication with the source of fluid; and
not in fluid communication with the inlet.

11. The valve assembly of claim 10, wherein, in a third position of the body, the second channel is aligned with the outlet such that the delivery conduit is in fluid communication with the source of fluid via the second channel.

12. The valve assembly of claim 11, wherein in the third position of the body, the channel is misaligned with the inlet and the outlet, such that the delivery conduit is not in fluid communication with the enclosure via the first channel.

13. The valve assembly of claim 9, wherein the body includes an insert positioned within the second channel, and wherein the insert is configured to inhibit the agent from moving through the second channel and permit the fluid to pass through the second channel.

14. The valve assembly of claim 1, further comprising a housing that defines the inlet and the outlet, the housing is configured to receive the body, wherein a gap is formed between the housing and the body, the gap is positioned between the inlet and the outlet such that the delivery conduit is in fluid communication with the source of fluid via the gap when the body is in the first position.

15. The valve assembly of claim 14, wherein the gap is sized such that the agent cannot pass through the gap.

16. A device for delivering an agent, comprising:
an enclosure configured to store the agent, the enclosure having an inlet;
a pressurized fluid source configured to store a pressurized fluid;
a valve assembly, including a body having a channel, wherein the body is configured to move between a first position and a second position to selectively fluidly couple the pressurized fluid source to the enclosure via the channel;
wherein, in the first position, the valve assembly is configured to misalign the channel from the inlet to inhibit the pressurized fluid from moving through the channel and delivering the agent out of the device; and
wherein, in the second position, the valve assembly is configured to align the channel with the inlet to permit the pressurized fluid from moving through the channel and delivering the agent out of the device.

17. The device of claim 16, wherein the body includes an insert positioned within the channel that is configured to inhibit the agent from moving through the insert and permit the pressurized fluid to pass through the insert.

18. The device of claim 16, wherein the channel is a first channel, wherein the body includes a second channel that is in fluid communication with the first channel;
wherein, in the first position and the second position of the body, the valve assembly is configured to inhibit the pressurized fluid from moving through the second channel and out of the device, and in a third position of the body, the valve assembly is configured to permit the pressurized fluid to move through the second channel and out of the device.

19. The device of claim 16, wherein the valve assembly includes a housing that is configured to receive the body, wherein a gap is formed between the housing and the body;
wherein, when in the first position, the valve assembly is configured to permit the pressurized fluid to move out of the device via the gap while inhibiting delivery of the agent out of the device.

20. A method for delivering a fluid from a medical device, the medical device including an enclosure for storing an agent, the method comprising:
moving a channel of a valve body to a first position that is aligned with an inlet and an outlet of the enclosure, thereby permitting delivery of a fluid through the channel and into the enclosure via the inlet to agitate the agent in the enclosure, and permitting delivery of the fluid and the agitated agent through the channel and toward a delivery conduit of the medical device via the outlet; and
moving the channel to a second position that is misaligned with the inlet and the outlet, thereby inhibiting delivery of the fluid through the channel and into the enclosure via the inlet, and inhibiting delivery of the fluid toward the delivery conduit via the outlet.
